(19) United States
(12) Patent Application Publication
(10) Pub. No.: US 2010/0042051 A1
(43) Pub. Date: Feb. 18, 2010

(54) System and Method for Holding a Catheter Near a Patient's Body

(76) Inventor: Donna Walker, North Kansas City, MO (US)

Correspondence Address: DAVID E HERRON II, PO BOX 12457, Overland Park, KS, 66282, US

(21) Appl. No.: 12/504,886

(22) Filed: Jul. 17, 2009

Related U.S. Application Data
(60) Provisional application No. 61/088,960, filed on Aug. 14, 2008.

(51) Int. Cl. A61M 25/02; A41B 9/06

(52) U.S. Cl. 604/174; 2/113

(57) ABSTRACT

The disclosure describes a system and method for use by a catheterized patient. A catheter having a first end inside a body of the patient passes through a stoma and terminates outside the patient. The disclosure also depicts a shirt having a pocket and an aperture positioned at a location between an area of a stoma (when the shirt is in the worn position) and the pocket cooperate to retain the terminus of the catheter.

### SUMMARY OF THE INVENTION

The invention is a system and method for use by a patient, particularly a patient requiring a catheter. While there are numerous procedures that employ catheterization of a patient, the invention herein is well-suited for patients requiring abdominal catheterization, such as those patients requiring peritoneal dialysis. Of course, the principles herein set forth can be applied equally well for patients requiring other types of catheters as well.

The inventive system will include a catheter having a first end inside a body of the patient. The catheter passes through a stoma, and terminates at a second end that is outside the patient. The system will also include a shirt with a shoulder portion and a lower portion configured to cover the patient's abdomen. The shirt will also have a pocket on an outer surface of the shirt, and an aperture positioned between the stoma (when the shirt is in the worn position) and an opening of the pocket. The catheter's second end (i.e., the free end) is retained within the pocket when the shirt is in a worn position.

Preferably, the shirt is an undergarment, such as a t-shirt or tank variety that can be worn beneath a shirt, sweater, or other garment. The inventive shirt assists in concealing the catheter from detection when worn as an undershirt. It is well-known that many catheterized patients are self-conscious about having detectable tubes come out of their bodies; a primary purpose of the inventive system is to provide a comfortable way of securing and concealing the catheter.

The invention may include a biasing means for urging the aperture toward a closed position. When the catheter passes through the aperture, the biasing means may grip and retain the catheter, thereby increasing the stability of the system. Preferably, the aperture (preferably formed as a button-hole like slit) in the shirt is positioned near a lower edge of a ribcage of the patient when the shirt is in the worn position.

In a preferred embodiment, the pocket is positioned near the patient's chest (when the shirt is in a worn position), and preferably retains the second end of the catheter. Preferably, the pocket forms an elongate rectangle with a longitudinal axis generally parallel to the patient's spine; however, alternate designs for the pocket are certainly within the scope of the invention. Specifically, the pocket is preferably formed so that it secures and retains the catheter.

The invention is also a method of retaining a catheter near a body of a patient. The method will include the step of inserting a catheter into a stoma of the patient so that a first end of the catheter is inside the patient and a second end is outside the patient and then providing a garment configured to engage and retain the second end of the catheter. The method will also include the step of positioning a pocket on an outward-facing surface of the garment, and making aperture on the garment.

Preferably, the aperture will be located between the stoma (when the shirt is in the worn position) and an opening of the pocket. This design and configuration enables a length of the catheter to pass between the garment and the body of the patient, then the catheter will pass through the aperture, and the catheter will terminate at a second end which is insertable into the pocket.

In a preferred embodiment of the inventive method, the garment is worn as a first layer of a multi-layered ensemble, much like an undershirt.

In a preferred embodiment, the aperture bears a button-hole like shape and bears a biasing means that urges the aperture toward a closed position. In a preferred embodiment, the aperture is formed near a lower edge of a ribcage of the patient when the shirt is in the worn position.

The invention also includes a shirt adapted to be worn by a patient using a catheter with a first end inside the patient and a second end outside the patient. The inventive shirt has a torso portion covering a stoma formed on the patient, and pocket positioned adjacent to (and preferably below) a breast portion of the shirt. The shirt will also include an aperture adapted to allow the catheter to pass through the shirt so that a portion of the catheter is inside the shirt and a terminal end of the catheter is outside the shirt. The aperture and pocket are positioned such that the catheter exits the stoma, passes through the aperture and terminates in the pocket.

The shirt works well when the stoma is on an abdomen of the patient, which represents the typical placement for patients requiring peritoneal dialysis. However, the principles of the invention also analogize to a patient with catheters in alternate positions, such as groin, leg, arm, armpit, chest, or the like.

Other objects, advantages and novel features of the present invention will become apparent from the following detailed description of the invention when considered in conjunction with the accompanying drawings.

### BRIEF DESCRIPTION OF THE DRAWINGS

FIG. 1 is a perspective view of a first embodiment of the inventive system, according to the principles of the invention.

FIG. 2 shows the embodiment shown in FIG. 1 in combination with a catheter.

FIG. 3 shows a perspective view of a second embodiment of the inventive system, according to the principles of the invention.

FIG. 4 shows the embodiment of FIG. 3 in combination with a catheter

FIG. 5 shows the inventive system, as it would be worn by a patient.

### DETAILED DESCRIPTION OF THE PREFERRED EMBODIMENTS

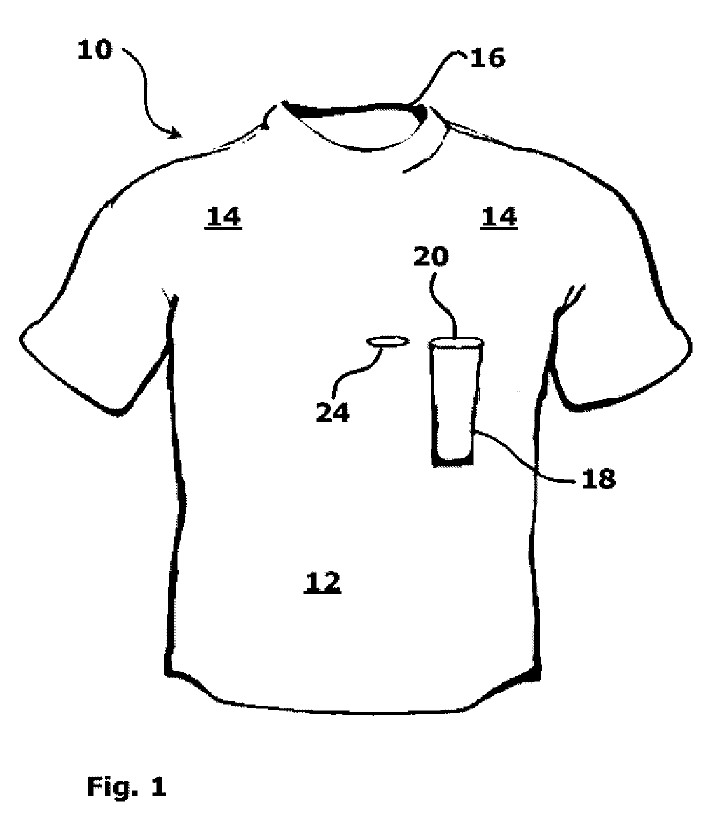

FIG. 1 presents a perspective view showing a portion of the inventive system 10, according to the principles of the invention. The system 10 includes a shirt having a torso portion 12, shoulder portions 14, and an open neck 16. The shirt also includes a pocket 18 near a breast portion of the shirt. Generally, the pocket 18 is shaped like an elongate rectangle with a longitudinal axis (i.e., length) that will be generally parallel the wearer's spine.

The invention depicted in FIG. 1 is well-suited to be worn by catheterized patients; indeed, the embodiment shown in FIG. 1 is best used by patients requiring peritoneal dialysis, which incorporates a catheter inserted into the peritoneal cavity at a stoma positioned approximately at the level of the navel, usually displaced 3-6 inches of it. An aperture 24 is formed in an area between an opening 20 of the pocket 18 and the approximate location of the stoma (not shown).

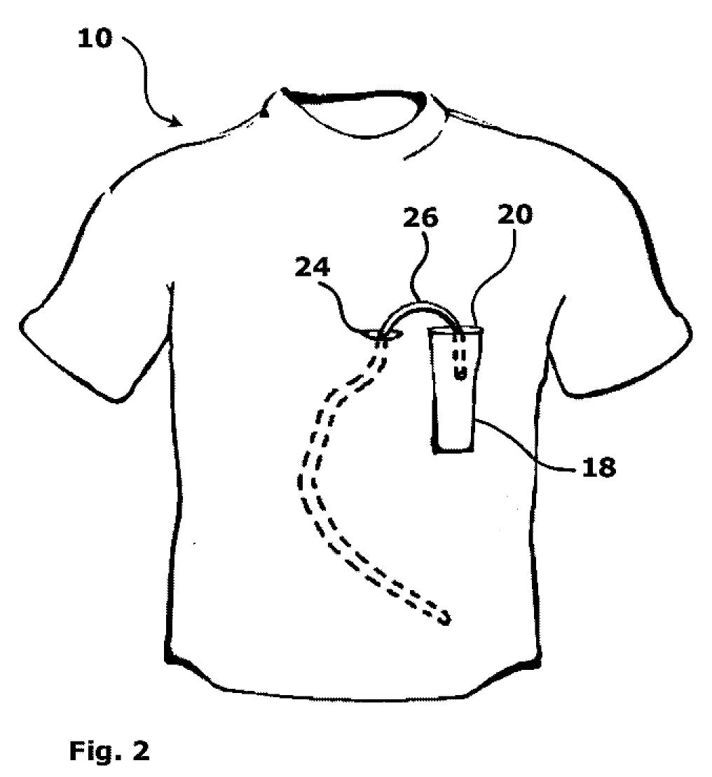

FIG. 2 shows the inventive system 10 in combination with a catheter 26 that originates within a patient's peritoneal cavity and comes from a stoma formed in the patient's abdomen. Generally, peritoneal dialysis requires a catheter of at least 12-24 inches in length. Typically, approximately 15-18 inches of tubing extends from the abdominal stoma of a dialysis patient. This length of tubing enables the patient to drain old fluid from the periloneal cavity (often into a commode or drain); also, this length conveniently also enables the patient to connect the end of the catheter to a fresh supply of fluid that can re-fill the peritoneal cavity.

As shown in FIG. 2, a length of the catheter 26 extends beneath the shirt (dotted portion) from the stoma in the patient to the aperture 24. The catheter 26 then passes through the aperture 24 so that a length of the catheter 26 is outside the shirt. The catheter 26 then passes through the opening 20 in the pocket 18; the terminal end of the catheter 26 is then held within the pocket 18.

FIG. 2 shows a system 10 that includes a T-shirt type of garment, such as a traditional undergarment or undershirt. This design keeps the catheter near tile patient's body and also conceals it beneath the patient's clothing. Of course, other undergarment styles (long underwear or tank-tops) are also within the scope and spirit of the invention.

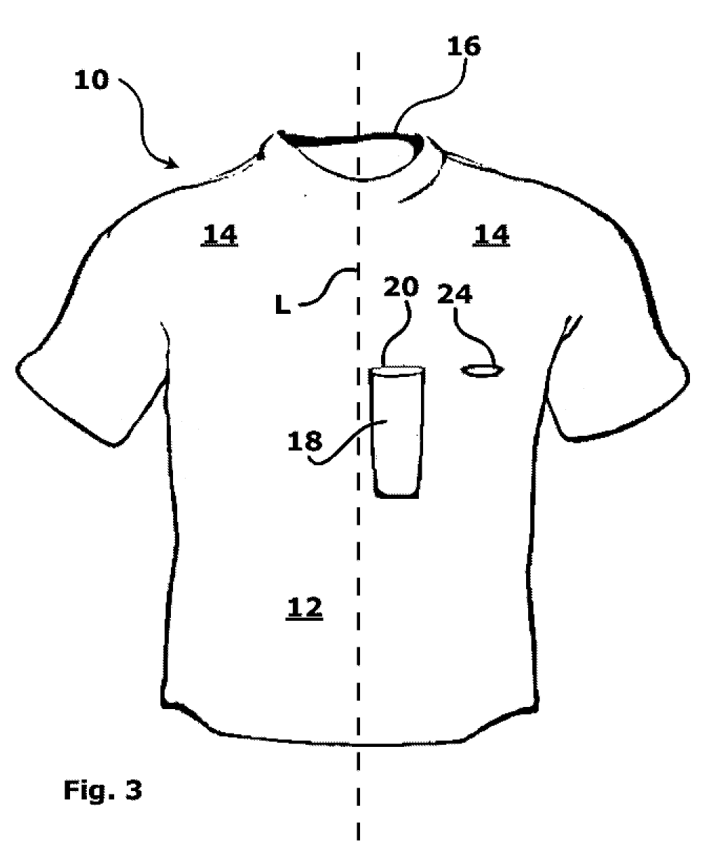

FIG. 3 shows a perspective view a portion of the inventive system 10, according to the principles of the invention. As in the previously-shown embodiment, the system 10 includes a shirt having a torso portion 12, shoulder portions 14, and an open neck 16 and a pocket 18 positioned near a breast portion of the shirt.

In the embodiment of FIG. 3, however, the relative positioning of the pocket 18 and aperture 24 are changed. In this embodiment, the aperture 24 is positioned distal a line of symmetry L of the shirt; in contrast, the pocket 18 is positioned proximate the line of symmetry L. This difference in aperture 18 and pocket 24 configuration (as from the embodiment shown in FIG. 1) provides an option that may provide increased comfort for many wearers. In this embodiment shown in FIG. 3, the catheter is typically guided away from the stomach area of the wearer while the catheter 26 is inside the shirt. The pocket 18 is preferably configured as an elongate rectangle with a length generally parallel the line of symmetry L.

In the embodiment shown in FIG. 3 (and the embodiment in FIG. 1 as well), the configuration of the pocket 18 is such that gravity helps retain the a catheter within the pocket 18. Additionally, the long, rectangular configuration frictionally grips and retains the catheter. Of course, other means of keeping the catheter secure within the pocket are within the scope and spirit of the invention.

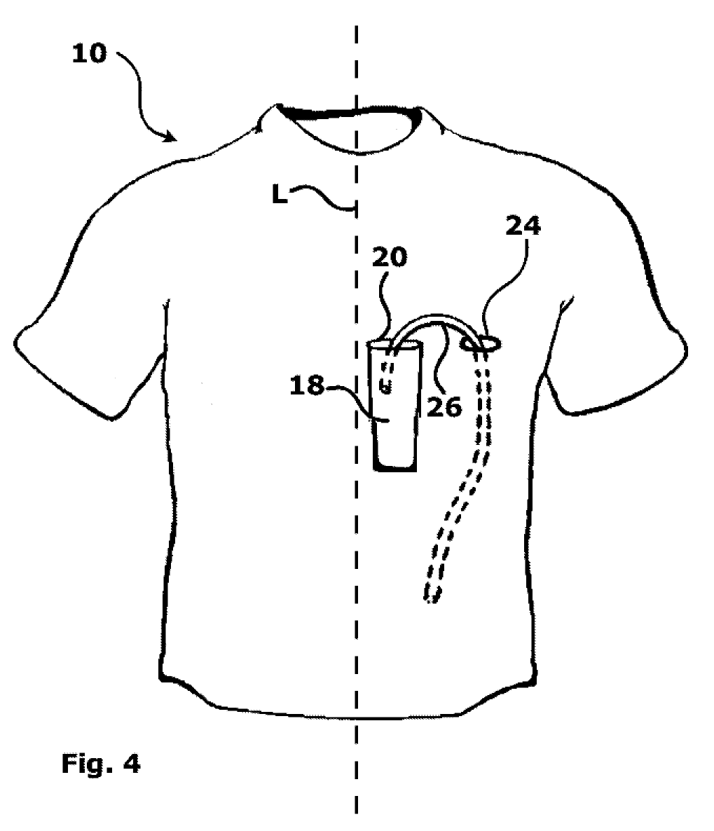

FIG. 4 shows the inventive system 10 in combination with a catheter 26 that originates within a patient's peritoneal cavity and comes from a stoma formed in the patient's abdomen. A length of the catheter 26 extends beneath the shirt (dotted portion) from the stoma in the patient to the aperture 24. The catheter 26 then passes through the aperture 24 so that a length of the catheter 26 is outside the shirt. The catheter 26 terminates within the pocket 18.

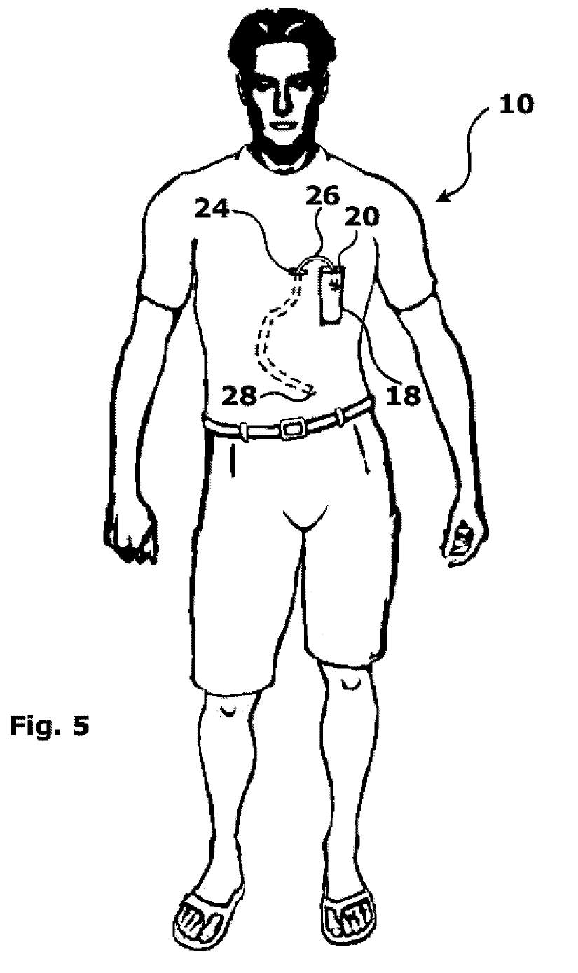

FIG. 5 shows the inventive system 10, as depicted in FIGS. 1 and 2, in use by a patient. The system 10 includes a shirt having a torso portion that extends at least to the patient's waist. When the lower portion is tucked into patient's pants, the system 10 provides a more secure means of keeping the catheter 26 inconspicuous and close to the wearer's body. The torso portion of the shirt covers a stoma 28 and a first length of the catheter 26, which first passes between the shirt and the patient, then passes through the aperture 24. The end of the catheter (capped, of course) is then retained within the pocket 18.

As shown in FIG. 5, the pocket 18 is preferably an elongate rectangle with a length generally parallel to the patient's spine, and is formed to be wide enough to enable easy insertion of the catheter 26, yet narrow enough to frictionally engage and retain the catheter 26. Additionally, because the shirt will be worn by active persons who are usually upright, gravity will also assist in keeping the terminal end of the catheter 26 within the pocket 18. Usually, the invention is worn beneath outerwear which will hide the catheter. Additionally, the wearer has options of wearing a more complete ensemble such as suspenders or vests.

Although the present invention has been described and illustrated in detail, it is to be clearly understood that the same is by way of illustration and example only, and is not to be taken by way of limitation. The spirit and scope of the present invention are to be limited only by claims that precisely define the metes and bounds of the invention.

My invention is:

1. A system for use by a patient, the system including:
a catheter having a first end inside a body of the patient, the catheter passing through a stoma and terminating with a second end outside the patient;
a shirt having
a shoulder portion;
a lower portion configured to cover the patient's abdomen;
a pocket on an outer surface of the shirt;
an aperture formed on a front of the shirt; wherein,
a length of catheter passes under the shirt, then passes through the aperture, and the second end of the catheter is retained within the pocket when the shirt is in a worn position.

2. The system as in claim 1, wherein the shirt is an undergarment.

3. The system as in claim 1, further comprising a biasing means for urging the aperture toward a closed position.

4. The system as in claim 1, the aperture positioned adjacent a lower edge of a ribcage of the patient when the shirt is in the worn position.

5. The system as in claim 1, wherein the pocket is configured to retain the second end of the catheter.

6. The system as in claim 1, wherein the pocket is positioned adjacent a patient's chest when the shirt is in the worn position.

7. The system as in claim 1, the pocket forming the shape of an elongate rectangle having a longitudinal axis generally parallel to the patient's spine.

8. The system as in claim 1, wherein the aperture bears a button-hole shape.

9. The system as in claim 1, wherein the pocket is positioned at a first distance from a line of symmetry of the shirt, and the aperture is formed a second distance from the line of symmetry of the shirt; wherein, the second distance is greater than the first distance.

10. A method for retaining a catheter near a body of a patient, the method including the steps of:
inserting a catheter into a stoma of the patient so that a first end of the catheter is inside the patient and a second end is outside the patient;
providing a garment configured to engage and retain the second end of the catheter proximate the patient;
positioning a pocket on an outward-facing surface of the garment;
positioning an aperture on the garment;
passing a length of the catheter between the garment and the body of the patient, and through the aperture; and,
inserting the second end of the catheter into the pocket.

11. The method as in claim 10, wherein the garment is an undershirt.

12. The method as in claim 10, further comprising the step of
Providing a means for biasing the aperture toward a closed position.

13. The method as in claim 10, the aperture positioned adjacent a lower edge of a ribcage of the patient when the shirt is in the worn position.

14. A shirt adapted to be worn by a patient using a catheter with a first end inside the patient and a second end outside the patient, the shirt comprising:
a torso portion covering a stoma formed on the patient;
pocket positioned adjacent a breast portion of the shirt;
an aperture adapted to allow the catheter to pass through the shirt so that a portion of the catheter is inside the shirt and the second end of the catheter is outside the shirt; wherein, the aperture and pocket are positioned such that the catheter exits the stoma, passes through the aperture and terminates in the pocket.

15. The shirt as in claim 14, wherein the aperture is positioned adjacent a lower edge of a ribcage of the patient when the shirt is in a worn position.

16. The shirt as in claim 14, wherein the shirt is an undershirt.

17. The shirt as in claim 14, wherein the stoma is on an abdomen of the patient.
